(19) United States
(12) Patent Application Publication
(10) Pub. No.: US 2018/0326035 A1
(43) Pub. Date: Nov. 15, 2018

(54) POTENTIATED T-CELL MODULATOR ABLE TO MODULATE IMMUNE RESPONSE, SPECIFICALLY DESIGNED FOR THERAPEUTIC USE AS A POTENTIATING ADJUVANT IN VIRUS VACCINES

(71) Applicant: HECTOR MANUEL ZEPEDA LOPEZ, Tlalnepantia (MX)

(72) Inventor: HECTOR MANUEL ZEPEDA LOPEZ, Tlalnepantia (MX)

(21) Appl. No.: 15/579,527

(22) PCT Filed: Jun. 4, 2015
(86) PCT No.: PCT/MX2015/000087

§ 371 Date: Dec. 4, 2017

(51) Int. Cl. A61K 39/12; A61P 31/14

(52) U.S. Cl. CPC A61K 39/12; A61K 2039/515; A61P 31/14

(57) ABSTRACT

Disclosed is a potentiated T-cell modulator (TCM) designed for treating the symptomology of the disease known as vitiligo. The TCM is obtained by means of a dialysable leucocyte extract from leucocyte cells that contain polypeptides equal to or less than 10,000 daltons, which extract comes specifically from Selachimorpha or shark spleen. The TCM can also reactivate the cells of an individual's immune system, enabling the organism to reactivate the cells responsible for skin pigmentation, i.e. melanocytes, and balance the production of melanin in the skin by means of this cell activation. The TCM has a potency of 10¹² leucocytes×mm³ (potency being understood as the quantity of leucocytes and quality of the flat, round and innocuous cells), which potency is the quantity necessary to excite the leucocytes and optimise the chemical signals for producing white blood cells, which in turn produce melanocytes, which produce melanin. When coupled to the chemical signals of neurotransmitters to potentiate the signal, the TCM helps the neurotransmitters to re-establish the chemical signals that travel from the brain to the bone marrow, establishing the correct chemical signals to the melanocytes and thereby “ordering” them to begin producing melanin.

### TECHNICAL FIELD

The present invention refers to a leukocyte extract containing polypeptides equal to or less than 10,000 daltons from shark spleen, to obtain a potentialized T-cell modulator (TCM for its acronym in English) capable of modulating the immune response through the activation of specific molecules involved in the control of innate immunity called “Toll-like Receptors” and its use as a pharmaceutically acceptable composition as an adjuvant in viral preparations used as vaccines ex. Influenza AH1N1. Likewise, the present invention relates to compositions comprising said T cell modulator and its combinations with viral antigens.

Similarly, the present invention also refers to a method for extracting and processing of a dialyzable leukocyte extract from selacimorphs for obtaining a potentiated T cell modulator specifically designed to be used as an adjuvant.

### BACKGROUND

The search for new substances with adjuvant activity is one of the most important trends in current immunological research. This with the objective of increasing the effectiveness of the immune response, since substances with immunostimulant properties are investigated for the potentialization of vaccines since the viruses become more resistant each day against the already developed vaccines, which translates into low effectiveness of the same.

The advances experienced in the last decade in peptide synthesis, nucleotide sequencing and the genetic engineering have made possible the development of the so-called “new generation vaccines”. However, the successful use of these new technologies requires a sustained research effort to find substances with adjuvant activity, effective in stimulating the immune response and devoid of adverse biological properties.

Adjuvants are chemical substances or preparations that, when are added to the antigen or injected simultaneously with it, make the immune response more effective. With its use an antigen and time economy is achieved, as well as a higher level of specific antibodies.

The free antigen normally diffuses very rapidly from the local tissues surrounding the inoculation site, and one of its important functions is to create a reservoir or deposit of the long-lived antigen. In the state of the art, it has been demonstrated that virtually all of the adjuvants activate or stimulate the immune system, mainly macrophages; these when activated stimulate the immune response by an increase in the amount of antigen expressed in the cell membrane and the efficiency of its presentation to lymphocytes. The macrophage also releases soluble stimulating factors, which amplify the proliferation of lymphocytes. On the other hand, some adjuvants have the ability to act specifically on lymphocytes; but, in general, they work better if they facilitate the simultaneous release of the antigen and immunomodulatory substances to the lymphoid tissue.

At the international level, the list of natural products and derivatives of chemical synthesis, with adjuvant/immunopotentiating properties, is increasing; however, only a small number is used in the formulation of human vaccines, there being a tendency related to the evaluation of new substances for this purpose.

In this sense, in the prior art there is the document: US 2009/0053197 TRANSFER FACTOR COMPOSITIONS AND METHODS, which mentions the possibility of using transfer factor as adjuvant, whose main feature is that the composition comprising transfer factor and an antibody, each is derived independently of a source selected from the group consisting of eggs, colostrum, blood, and combinations thereof. This means that the leucocyte extract has as source eggs, colostrum, blood or combinations thereof. However, for a person skilled in the art, it is well known that this patent presents the following problems, if egg yolk is used, it is very difficult to eliminate the lipids it contains, likewise, both the egg yolk, and the other sources consist of a low amount of leukocytes×mm³, which reduces the power to said invention. Likewise, the synthesis of the previous sources of leukocyte extract is very complicated to separate leukocyte cells from proteins, lipids, carbohydrates and toxins.

For example, the patent document WO2002/024746. TRANSFER FACTOR FROM BIRD EGGS. Said document uses a method to obtain a transfer factor from the egg yolk of animals, however, a person skilled in the art knows that the method to extract transfer factor from eggs is very complicated, especially in the separation of lipids. Likewise the units of transfer factor obtained by egg is thousands of times less than the present invention.

Likewise, the document U.S. Pat. No. 4,816,563. PROCESS FOR OBTAINING TRANSFER FACTOR FROM COLOSTRUM, TRANSFER FACTOR SO OBTAINED AND USE THERE OF; uses as a source of extraction of the transfer factor the colostrum of animals, specifically mammals. However, like the previous document, the amount of transfer factor units obtained from colostrum is minimal compared to the present invention, since the TCM found in sharks is 10¹² leukocytes×mm³, against 10⁶ leukocytes×mm³ of the colostrum.

In the same way, the document WO 2005/028622. COMPOSITIONS CONTAINING DIFFERENT TYPES OF TRANSFER FACTOR, METHODS FOR PREPARING COMPOSITIONS AND METHODS OF TREATMENT USING THE COMPOSITIONS. This document combines a composition to produce an immune response mediated by T cells in an individual, which contains the transfer factor of at least two different types of animals. For example, the composition may contain the mammalian transfer factor and a non-mammalian transfer factor. An example of the composition may be a combination of a colostrum-derived product, which includes the mammalian transfer factor, and an egg-derived product, which includes the non-mammalian transfer factor. Like the previous ones, this document presents two problems, eliminating the lipids contained in the egg yolk and the low amount of leukocytes×mm³, which reduces the power to said invention.

In the same way, it was found that the patent MX 9504215 IMPROVED PROCEDURE FOR THE PURIFICATION OF OLIGOPEPTIDES WITH MOLECULAR WEIGHTS OF 1000 TO 10,000 DALTONS, FROM LYMPHOCYTE STRIPS AND ITS PHARMACEUTICAL PRESENTATION, this document refers to an improved procedure for fractionation with high yield, from a set of oligopeptides (from 1000 to 10000 daltons), recovered from the breakdown of leukocytes and possessing biological activity for the regulation of the immune response comprising the following steps: from leukocytic packages of healthy donors, ALL UNDER ASEPTIC CONDITIONS, the cells are broken, suspensions are made adjusting volumes, and by ultracentrifugation, the suspension of cellular debris is clarified (cellular detritus), the oligopeptides are recovered by means of diafiltration and concentrated by tangential ultrafiltration. The product is formulated based on its formula of finished product in pharmaceutical presentation. However, a person skilled in the art knows that the use or utilization of human blood and its distilled derivatives or components separated from chemical procedures, cannot be commercialized, since different legislations in the world forbid it and it is classified as a crime the marketing of human blood and its derivatives. Likewise, the units of transfer factor obtained for each 450 ml of blood of healthy donors of the present invention, although is higher than the units of transfer factor obtained from egg yolk and colostrum, is only 10⁸ leukocytes×mm³.

Likewise, the document WO 97/12915, PROCEDURE FOR PURIFICATION OF THE TRANSFER FACTOR FROM LEUKOCYTES; denotes a process of purification of the Transfer Factor (oligopeptides of 1,000 to 10,000 daltons, which possess biological activity), from leukocytes, which comprises the following steps: the cells are lysed in sterile conditions, the suspension is clarified by ultrafiltration, the Transfer Factor is recovered by diafiltration and is concentrated by tangential ultrafiltration. The Transfer Factor is used pharmaceutically as a regulator of the immune response. However, this document is related with the document MX 9504215 and has the same problem of using human blood as a leukocyte extraction medium.

The patent MX20089296A, OPTIMISED PROCESS FOR THE OBTENTION OF DIALYZABLE LEUKOCYTE EXTRACT, CONTAINING PEPTIDES WITH MOLECULAR WEIGHT EQUAL TO OR LOWER THAN 10,000 DALTONS, FROM CROCODILE LYMPHOID TISSUE AND THE PREPARATION THEREOF IN AN ORAL AND/OR INJECTABLE PHARMACEUTIC, refers to a optimized procedure for obtaining the leukocyte extract containing peptides with molecular weight equal to or less than 10,000 Daltons, from cells of lymphoid tissue from crocodile and which has the property of regulating the immune response in humans and animals. However, the power of the transfer factor obtained is 10⁸ leukocytes×mm³.

Finally, it is mentioned the documents: WO2008/042604. IMMUNE MODULATORS, PREPARATIONS AND COMPOSITIONS INCLUDING IMMUNE MODULATORS, TESTS FOR EVALUATING THE ACTIVITY OF IMMUNE MODULATORS AND PREPARATIONS AND COMPOSITIONS INCLUDING THE SAME, AND METHODS. Which discloses compositions including extracts from sources of immune modulators that include the immune molecules of the nanofraction modulator (ie, molecules that have molecular weights of about 3,000 DA and less). These compositions may also include other immune modulators, such as transfer factor. U.S. Pat. No. 4,468,379, LEUKOCYTE EXTRACTS FOR AFFECTING THE IMMUNE SYSTEM. Which describes a group of substances derived from human leukocytes (white blood cells) that amplify, suppress, or otherwise modulate the response of the immune system to the reintroduction of antigens. And U.S. Pat. No. 5,840,700, METHODS OF PRODUCING TRANSFER FACTOR. Which discloses that the invention relates to the substantially pure transfer factor with a specific activity of at least 5000 units per unit of absorption at 214 nanometers. The current invention also relates to a process for preparing the cell lysate transfer factor. The present invention includes the use of the substantially pure transfer factor with a specific activity of at least 5000 units per unit of absorption at 214 nm to treat infectious diseases.

Therefore, it is evident that a leukocyte source is not available for the production of potentiated TCM and without adverse effects. It has been tried to use transfer factor from egg yolk, milk and blood, wherein although the product acts as an immunomodulator, there is a low concentration of leukocytes in said sources, which translates into a low cellular excitation and deficient coupling to chemical signals. Also, these sources for extracting leukocyte extract, when administered, can cause allergic reactions to patients, such is the case of the egg. All of the above, although with different methods of synthesizing the leukocyte extracts, use as a leukocyte source either eggs of mammals, fish or birds, colostrum, blood or their combinations. However, for a person skilled in the art, it is well known that prior patents generally present the following problems, if egg yolk is used, it is very difficult to eliminate the lipids it contains, likewise, both the yolk egg, as the other sources consist of a low amount of leukocytes×mm³, which reduces the power to said invention. Likewise, the synthesis of the previous sources of leukocyte extract is very complicated to separate leukocyte cells from proteins, lipids, carbohydrates and toxins. In the same way, it is important to mention that various legislations in the world prohibit the commercialization of human blood and their derivatives. No invention was found in the prior art that refers to a method for the leukocyte counting and verification of the activated chemical signals of the leukocyte extract.

Is therefore, of particular interest the evaluation of new immunoadjuvants to increase the effectiveness of vaccines. That is why the present invention proposes to use as an adjuvant a leukocyte dialyzable extract whose source are sharks.

In the year of 1955, Lawrence, interested in specific active immune memory, found that this could be transferred from one person to another by means of a leukocyte extract injection to which he called ‘Transfer Factor’.

Transfer Factor: leukocyte dialyzed extract constituted by a group of molecules of low molecular weight between 1.0 and 6.0 KDa; these molecules store the exclusive immune experience of the animal which, in turn, can be transferred to the human.

Nevertheless, in the prior art there is not any means for obtaining a dialyzable extract of leukocytes containing polypeptides less than or equal to 10,000 Daltons whose source or origin is from cells, tissues or organs of sharks, more specifically shark spleen, of the present invention, is considered novel, since when the prior art was analyzed, methods were found for said extraction of leukocytes, white cells or T cell modulator, by means of leukocytic packages of healthy donors (humans); eggs of reptiles, amphibians, fish and birds; besides colostrum (milk produced by mammal) and crocodile spleen. However no document was found which mentioned the possibility of extracting leukocyte cells from selacimorphs for the extraction of TCM, which are a superorder of chondrichthyes (cartilaginous fish) commonly known as sharks, wherein it has been demonstrated in in vitro tests to be eight times more powerful than the peripheral blood derivative of healthy donors in the induction of cytokines, specifically IL-6, IFN-g and TNF-a.

In this sense, the concept about the immunological capacity in ancestral animals is: The longer age the immune system is better, which allows to survive so many years, so when giving us the task of obtaining spleen cells from an ancestral animal it was found that the Shark is an excellent candidate to obtain spleen cells that can be used to obtain a higher potency T-cell modulator.

Therefore, numerous clinical trials have been carried out in the last 3 decades using extracts of white blood cells known as “transfer factor”. Unfortunately, the different scientific groups working in this field were not able to consistently generate purified and reproducible preparations of the transfer factors. Despite this, the efficacy of transfer factors have been published in revised publications for the treatment of conditions ranging from multiple sclerosis, cancer, and herpes infections, but none for the treatment of vitiligo.

In this sense, the inventors of the present invention were able to develop an economic and scalable protocol for the generation of an optimized transfer factor from shark leukocytes. Unlike previous versions of transfer factors, TCM is characterized at molecular level and its main means of inducing therapeutic effects have been elucidated.

According to the previous, it is evident that at present there is no an effective treatment available against the destruction of the melanocytes that occurs in vitiligo. Repigmentation has been attempted through the use of steroids or immunomodulators (topical and systemic) with poor results, the psoralens in combination with ultraviolet light sessions are also used with limited efficacy, all of these without achieve fully satisfactory results.

Therefore, in the prior art there is no source that provides a dialyzable extract of leukocytes containing polypeptides less than or equal to 10,000 Daltons whose source or origin is from cells, tissues or organs of sharks with a concentration of T cells modulator of around 10¹² leukocytes×mm³ for use in the treatment of vitiligo. Since, in said prior art, the T cell modulator concentrations of the known extraction media, ranges from 10⁴ to 10⁸ leukocytes×mm³.

### Objects of the Invention

Therefore, it is an object of the present invention to provide a source of leukocyte extract whose origin is not from mammals, eggs, nor colostrum, and which provides a potency of 10¹² leukocytes×mm³ (understanding by potency to the amount of leukocytes and quality of smooth, round and innocuous cells), necessary amount for cellular excitation and optimization of chemical signals.

Another object of the present invention is to provide a potentiated T cell modulator with a concentration of approximately 10¹² leukocytes×mm³ as an adjuvant in viral preparations to potentiate vaccines.

It is also an object of the present invention to provide a potentiated T cell modulator with a concentration of approximately 10¹² leukocytes×mm³, and its compositions and pharmaceutical formulations specifically designed to be used as an adjuvant.

Yet another additional object of the present invention is to provide a potentiated T cell modulator with a concentration of approximately 10¹² leukocytes×mm³ with adjuvant activity, effective in stimulating the immune response and devoid of adverse biological properties.

A further object of the present invention is to provide, it relates to a leukocyte extract containing polypeptides equal to or less than 10,000 daltons from shark spleen origin and its use as immunomodulator adjuvant in viral preparations used as vaccines.

### BRIEF DESCRIPTION OF THE FIGURES

FIG. 1 shows the method of checking the power of the leukocyte extract from the inoculation of the leukocyte extract in Balb-c mice.

FIG. 2. Graphs IgM antibodies against Influenza p/AH1N1.

FIG. 3. Graphs IgC antibodies against Influenza p/AH1N1.

FIG. 4. Graphs PCR results in TCM of test 1.

FIG. 5. Graphs PCR results in TCM of the test.

### DETAILED DESCRIPTION OF THE INVENTION

The present invention relates in general to compositions comprising a T-cell modulator (TCM), whose source or origin is from the shark spleen, particularly the present invention refers to a T-cell modulator capable of modulating the response immune through the activation of specific molecules involved in the control of innate immunity called “Toll-like Receptors” and its use as a pharmaceutically acceptable composition as an adjuvant in viral preparations used as vaccines ex. Influenza AH1N1. Also, the present invention relates to compositions comprising said T-cell modulator and its combinations with viral antigens, and to compositions comprising said T-cell modulator in conjunction with antigens. Such compositions are useful in the prevention and/or treatment of diseases.

The present invention also relates to a dialyzable extract of leukocytes containing polypeptides less than or equal to 10,000 Daltons whose source or origin is the shark spleen, which has a concentration of T cell modulator of about 10¹² leukocytes×mm³ for use as an adjuvant by isolating the appropriate cytosine(s) for each type of vaccine to be administered to the patient, and according to the type of virus.

As mentioned above, the transfer factors, which are leukocyte extracts produced by the leukocytes, which were previously induced by an immunization ie, are amino acids that stimulate and transfer the immunity transmitted by cells from one individual to another and through one species to another, while the T cell modulator (TCM) is found naturally without being induced by an immunization, therefore it is different to the transfer factor, in addition said TCM does not create contraindicated effects or known harmful responses.

The prevention of infections and/or diseases from vaccines combined with the potentiated T-cell modulator of the present invention increases the effectiveness thereof since it promotes cellular excitation to obtain an acquired immunity.

The T cell modulator is obtained from a dialyzable extract of the cells contained in the shark spleen and containing extracellular fluid. This facilitates antigen-antibody recognition and strengthens the chemical bond preventing ruptures thereof.

In the state of the art, the antigen-antibody chemical bond may not be performed or do not exist. However, with the adjuvant of the leukocyte extract from a TCM from shark spleen of the present invention, it is favored the presentation of the antigen, from the functional point of view (the TCM functions as a chemical signal that attracts the host-presenting cells of antigen like the dendritic cells and the macrophage) and also from the morphological point of view by increasing the molecular weight of the antigen and being more easily recognized by the macrophage, the lymphocytes and the T-B relationship is more efficient and the antigen-lymphocyte B chemical bond, in addition it is strengthened, and is done in less time.

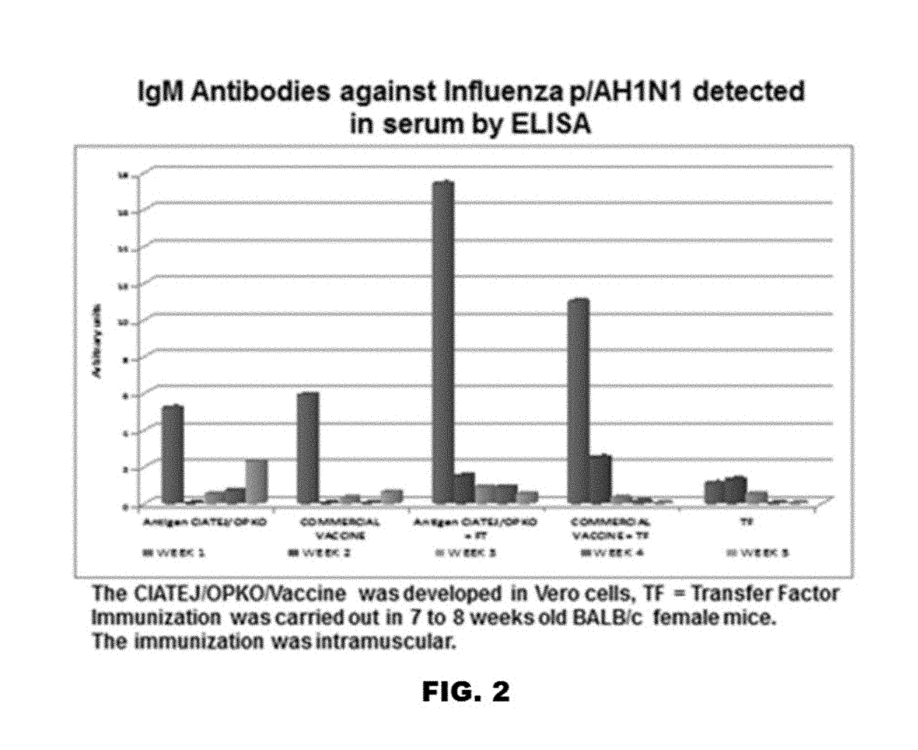

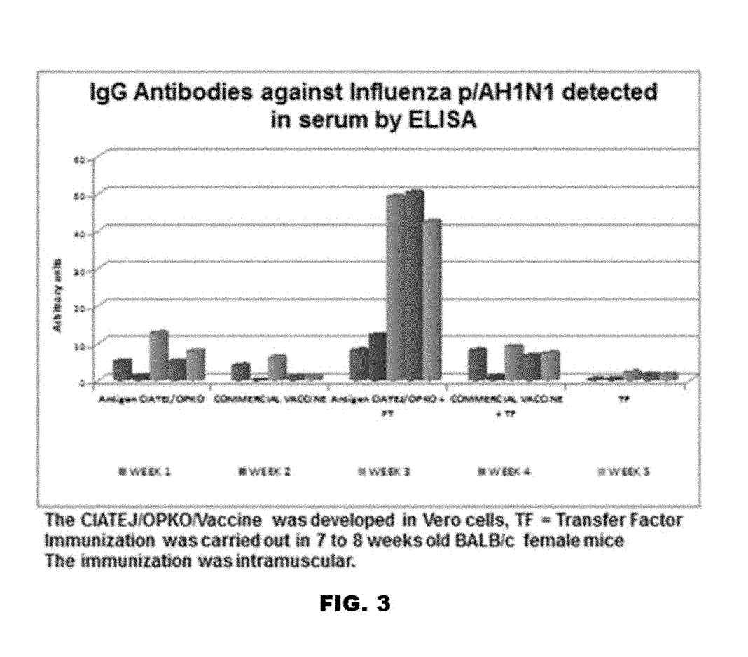

Therefore, the TCM of the present invention whose source is a specific zone of the spleen that is part of the lymphatic system and is the center of activity of the immune system of selacimorphs, is rich in B lymphocytes that produce chemical signals that when interacting with the cooperative T lymphocytes, have a positive collaboration for the synthesis of antibodies, that being linked the chemical signals with the viral antigen of the vaccine generates a greater quantity of antibodies directed against the viral antigen of the vaccine, additionally that the Immunological response is faster expressed by the production of IgM and lasting expressed by the production of IgG (FIGS. 2 and 3).

Likewise, the leukocyte extract of the present invention gives greater durability to the formed chemical bond antigen-lymphocyte B.

Therefore, the T cell modulator obtained from a dialyzable extract of the cells contained in the shark spleen and containing extracellular fluid, functions as immunomodulatory adjuvant T-cell modulator, since it is known in the state of the art for a person skilled in the art, that the function of a T cell modulator is to increase cellular excitation, as well as to improve the chemical signals for the production of white blood cells.

As mentioned above, the common sources of transfer factors are colostrum, egg yolk, human blood, etc. However, the T-cell modulator units obtained from the above sources are much smaller than those obtained in the present invention, that is, about 10⁶ leukocytes×mm³. Therefore, it should be noted that immunomodulatory adjuvant T-cell modulator from shark spleen of the present invention, consists of a potency of 10¹² leukocytes×mm³ (understanding by potency the amount of leukocytes and quality of smooth, round and innocuous cells)), necessary amount for the excitation of leukocytes and optimization of chemical signals for the production of white blood cells.

One of the specific effects of the T-cell modulator is a significantly increased activity of NK (natural killer) cells. NK cells provide protection against viruses as part of the natural immune defense system.

Additional aspects of the invention relate to compositions and formulations which may comprise components in presentation such as powder, encapsulated, including, but not limited to, encapsulated T cell modulator or powder and/or antibody or encapsulated fraction of the antibody.

Additional aspects of the present invention are directed to the methods of compositions and formulations of manufacture according to the vaccine.

In this sense, it is another aspect of the present invention, the methods of treating and/or preventing certain conditions comprising the administration of an effective amount of a composition and/or a formulation comprising T-cell modulator.

Extraction and processing method of leukocyte extract specifically designed as adjuvant

The process of the present invention for obtaining a dialyzable extract of leukocytes containing polypeptides less than or equal to 10,000 Daltons whose source or origin is from shark cells, tissues or organs, more specifically shark spleen, specifically designed as immunomodulatory adjuvant, is the following:

Sterilization.—Involves that any instrument used to extract the leukocyte extract should be sterile.

Extraction of spleen. This step consists of extracting the shark spleen surgically in order to extract the leukocyte extract, from the surgical extraction of a specific area of the shark spleen, rich in B lymphocytes, which when administered to the individual, produces chemical signals which when interacting with the cooperative lymphocytes T, have a positive collaboration for the synthesis of antibodies, which by being linked the chemical signals with the viral antigen of the vaccine generates a greater amount of antibodies directed against the viral antigen of the vaccine, additionally that the immune response is faster expressed by the production of IgM and durable expressed by the production of IgG.

Counting and quantification.—By means of the Neubauer chamber and microscope, the number of leukocytes per field in the Neubauer grid is counted, to know the power of the T-cell modulator that will be obtained, that is, the number of leukocytes per cubic millimeter. Likewise, it is necessary to evaluate the quality of said cells, so that there is no anisocytosis, that is, through the microscope it is observed that the cells are round and smooth.

Breaking or separation of components.—The leukocyte cells must be separated from proteins, lipids, carbohydrates and toxins.

Dialysis.—Dialysis is the process of separating the molecules in a solution by the difference in their diffusion rates through a semipermeable membrane. Then, the leukocyte extract, after the separation and breaking of components has been performed, is placed in a semipermeable dialysis bag, for example, in a membrane of the cellulose with pores, and the bag is sealed. The sealed dialysis bag is placed in a vessel with a different solution, or pure water. Due to the fact that the leukocyte extracts, is small enough to pass through the pores tend to move inwards or outwards of the dialysis bag in the direction of the lowest concentration. The larger molecules (often proteins, DNA, or polysaccharides) which have dimensions significantly larger than the pore diameter are retained within the dialysis bag. In this way, leukocyte extracts less than or equal to 10,000 Daltons are separated.

Filtration and sterilization.—After the dialysis, the leukocyte extract is filtered by means of a membrane of pore size between 2 and 4 micrometers. Likewise, the solution is sterilized again.

Formulation.—A lyophilization process is carried out to remove the water from the leucocyte extract by means of the generation of a vacuum, likewise in this step the aggregation of a vehicle is carried out.

Physical-chemical evaluation. At this point the physical-chemical processes such as density, pH, color, smell and taste are evaluated. It is important to mention that if no vehicle was added to the product, the T-cell modulator obtained will be odorless, colorless and tasteless.

Evaluation of biological activity. The required cytosine(s) for its function as an adjuvant are induced, this from the dilution of the leukocyte extract until reaching the title of the required cytokines depending on the type of vaccine to which the leukocyte extract will act as an adjuvant. This means that the cytosine(s) suitable for functioning as an adjuvant of the present invention will be chosen according to the type of vaccine to be administered to the patient, in such a way that the required cytosine(s) will be isolated according to the vaccine, such is the case of the AH1N1 viruses, among others. Equally, the leukocyte extract will be also analyzed with microarray membranes to determine the type of cytosine found in the leukocyte extract.

The result of the previous process is a potentiated T-cell modulator in powder form, which provides it the virtue of being easily transported and stored, it does not require refrigeration and a power of T cell modulator of 10¹² leukocytes×mm³ is obtained, which is highly superior to any known T-cell modulator, being understood by potency the concentration of leukocytes per mm³ and the quality of the cells (smooth, round and innocuous).

The present figure illustrates the type of cytokines induced from the inoculation of the leukocyte extract in Balb-c mice. Groups of 8 mice are used, which will be inoculated with the amount equivalent to the weight-unit relationship of T-cell modulator (0.005 unit of T-cell modulator).

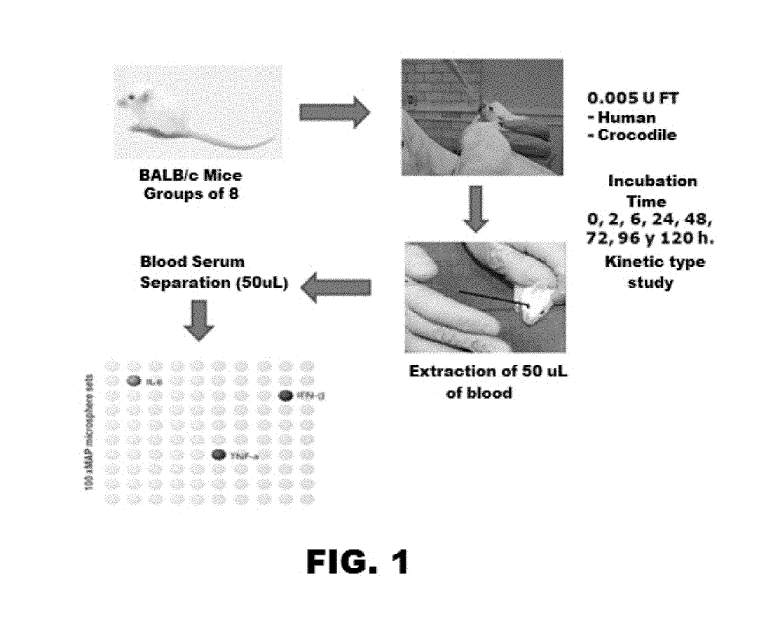

According to FIG. 1 serum is extracted from each mouse it is used 50 microliters of serum, they are exposed to the microarray membranes containing the receptor antibodies of the cytokines and the wells that develop color will be the induced cytokines. The serum is diluted with a regulating solution in multiples of 2 initially so as to subsequently carry out dilutions in multiples of 100. The basal dilution of time 0 is eliminated and the dilution that preserves the color development in the microarrays prior to dilution where it is no longer present the color development, is the title of the leukocyte extract. In this way, suitable cytokines can be isolated to adjuvate the required viral vaccine.

| CITOKINE | CITOKINE |
|---|---|
| EOTAXIN | IL-15 |
| G-CSF | IL-17 |
| GM-CSF | IP-10 |
| INF-γ | MIP-2 |
| IL-1α | KC |
| M-CSF | LIF |
| IL-1β | LIX |
| IL-2 | MCP-1 |
| IL-3 | MIP-1α |
| IL-4 | MIP-1β |
| IL-5 | MIG |
| IL-6 | RANTES |
| IL-7 | TNFα |
| IL-10 | IL-12 (P70) |
| IL-12 (p40) | VEGF |
| IL-13 | IL-9 |

Evidence:

56 pigs of approximately 20 kg were selected.

Twenty-eight pigs were used for control group and test group respectively, placed in pens of 14 pigs each.

At the beginning of the evaluation, blood samples were taken for Real Time PCR diagnosis and corroborate the presence and quantification of the PRRS virus.

The test group was given 5 ml of T-cell modulator and the control group was given 5 ml of saline solution as a placebo.

After 10 days, blood sampling was repeated for PCR TR analysis in order to observe the dynamics of virus infection.

The mortality of both groups was recorded.

### PCR Results

Sampling before the application of FT and SSF: Nov. 25, 2012

| Identification: | Group: | Result: | Quantification: | Viral load |
|---|---|---|---|---|
| 1, 2, 5 | FT | Positive | * 7.99 × 10³ | 7990 |
| 23, 24, 25 | Controls | Positive | * 1.63 × 10⁴ | 16400 |

### PCR Results

Sampling after the application of FT and SSF: Dec. 5, 2012

| Identification: | Group: | Result: | Quantification: | Viral load |
|---|---|---|---|---|
| 1, 2, 5 | FT | Positive | * 1.45 × 10³ | 1450 |
| 23, 24 | Controls | Positive | * 3.45 × 10⁴ | 34500 |

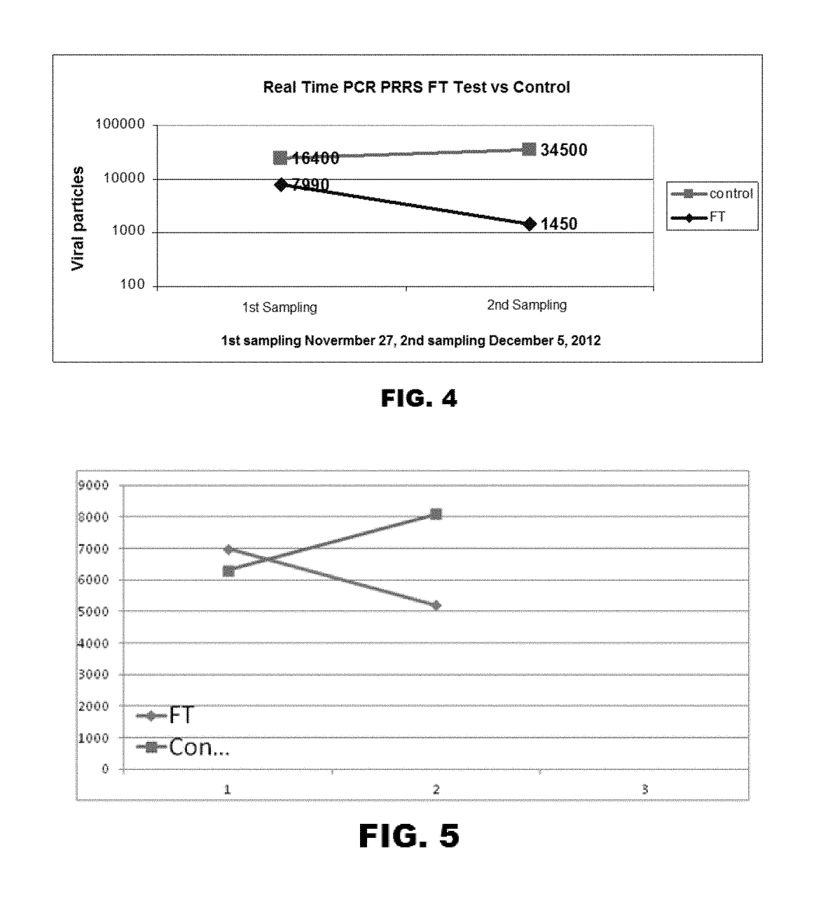

The PCR Results at TR of Test 1 are Shown in FIG. 4.

### CONCLUSIONS

The group treated with FT shows a considerable reduction of the viral load, in comparison with the control group, nevertheless, it is necessary to run evaluations in which we can determine with a statistical model the reliability of the results.

### Test 2

60 pigs of 20 kg approximately were selected and 45 days old.

30 pigs were used for control group and 30 for group treated with TF, were placed in pens of 15 pigs each.

At the beginning of the evaluation, blood samples were taken for Real Time PCR diagnosis and corroborate the presence and quantification of the PRRS virus.

The test group was given 5 ml of T-cell modulator and the control group was given 5 ml of saline solution as a placebo.

After 10 days, blood sampling was repeated for PCR TR analysis in order to observe the dynamics of virus infection.

The mortality of both groups was recorded.

### PCR Results

Sampling before the application of FT and SSF: Feb. 25, 2013

| Identification: | Group: | Result: | Quantification: | Viral load |
|---|---|---|---|---|
| 6, 8, 10 | FT | Positive | * 6.99 × 10³ | 6990 |
| 16, 17, 18 | Controls | Positive | * 6.30 × 10⁴ | 6300 |

### PCR Results

Sampling after the application of FT and SSF: Dec. 25, 2012

| Identification: | Group: | Result: | Quantification: | Viral load |
|---|---|---|---|---|
| 6, 8, 10 | FT | Positive | * 5.20 × 10³ | 5200 |
| 16, 17, 18 | Controls | Positive | * 8.10 × 10³ | 8100 |

The PCR Results at TR of Test 2 are Shown in FIG. 5.

1-14. (canceled)

15. Potentiated T cell modulator (TCM) designed to function as an adjuvant in viral preparations to potentiate vaccines, said TCM is characterized in that: it is obtained by means of a dialyzable extract of leukocytes from leukocyte cells containing polypeptides equal to or less than 10,000 daltons, whose specific source is shark spleen, which is part of the lymphatic system and is the center of activity of the immune system of said sharks, said shark spleen is rich in B lymphocytes that produce chemical signals which when interacting with the cooperative T lymphocytes, have a positive collaboration for the synthesis of antibodies, that by being linked the chemical signals with the viral antigen of the vaccine generates a greater quantity of antibodies directed against the viral antigen of the vaccine, further causing that the immune response is faster expressed by the production of IgM and durable expressed by the production of IgG, wherein said TCM has a power of 1012 leukocytes×mm3 (understood by power the amount of leukocytes and quality of smooth, round and innocuous cells), whose power is the necessary amount for the excitation of leukocytes and optimization of the chemical signals for the production of white blood cells.

16. Method of extracting, checking and counting leukocyte dialyzable extract from leukocyte cells containing polypeptides equal to or less than 10,000 daltons, whose specific source is a zone of the shark spleen, rich in B lymphocytes, which is part of the system lymphatic and is the center of activity of the immune system of said sharks, to obtain a potentiated T-cell modulator specifically designed as an adjuvant, comprising the following steps: sterilizing any instrument used to extract the leukocyte extract; extraction of the leukocyte extract from surgical extraction of a specific area of the shark spleen, rich in B lymphocytes; counting and quantificating by means of the Neubauer chamber and microscope, the number of leukocytes per field in the Neubauer grid is counted, to know the power of the T-cell modulator that will be obtained, that is, the number of leukocytes per cubic millimeter; evaluating the quality of said cells, so that there is no anisocytosis, that is, through the microscope it is observed that the cells are round and smooth; separating the leukocyte cells from proteins, lipids, carbohydrates and toxins; separating the molecules in a solution by the difference in their diffusion rates through a semipermeable membrane; placing the leukocyte extract, after having been separated in a semipermeable dialysis bag, as for example, in a cellulose membrane with pores, and sealing the bag; placing the sealed bag in a container with a different solution, or pure water, where the leukocyte extracts, being small enough to pass through the pores tend to move in or out of the dialysis bag in the direction of the lowest concentration, larger molecules (often proteins, DNA, or polysaccharides) that have dimensions significantly larger than the pore diameter are retained within the dialysis bag, separating leukocyte extracts less than or equal to 10,000 Daltons; filtering the leukocyte extract by means of a membrane of pore size between 2 and 4 micrometers; sterilizing the solution again; performing a lyophilization process to remove water from the leukocyte extract by generating a vacuum, and performing the aggregation of a vehicle; evaluating the physical-chemical processes such as density, pH, color, smell and taste, wherein if the product is not added with any vehicle, the obtained T cell modulator will be odorless, colorless and tasteless; said method is characterized in that it comprises the steps of: inducing the cytosine (s) required for its function as an adjuvant, this from the dilution of the leukocyte extract up to reach the title of the required cytokines according to the type of vaccine to which the leukocyte extract it will act as an adjuvant, wherein the cytosine (s) suitable for functioning as an adjuvant of the present invention will be chosen according to the type of vaccine to be administered to the patient, so that the cytosine (s) required according to the vaccine will be isolated; and analyzing the leukocyte extract with microarray membranes to determine the type of cytosine found in the leukocyte extract.

17. The potentiated T cell modulator according to claim 1, wherein it isolates the cytokine (s) suitable for its function as an adjuvant for each type of vaccine that will be administered to the patient, according to the type of virus.

18. The potentiated T cell modulator according to claim 1, wherein it increases the effectiveness of the vaccines since it promotes cellular excitation to obtain an acquired immunity.

19. The potentiated T-cell modulator according to claim 1, wherein it facilitates the antigen-antibody recognition and strengths the chemical bond preventing ruptures thereof.

20. The potentiated T cell modulator according to claim 5, wherein the chemical bond antigen-lymphocyte B, besides being strengthened, is performed in less time.

21. The potentiated T cell modulator according to claim 1, wherein the obtained product is in powder form, which can be easily transported and stored, also does not require refrigeration.

22. The potentiated T cell modulator according to claim 7, wherein the product obtained also comprises compositions and formulations in powder form, encapsulated, including, but not limited to, encapsulated T cell modulator or powder and/or antibody or encapsulated fraction of the antibody.

23. The method according to claim 2, wherein the power of the leukocyte extract is checked from the inoculation of the leukocyte extract in Balb-c mice, wherein the group of mice that retains the maximum title with longer induction time will be the time of induction of the cytosine induction.

24. The method according to claim 10, wherein the higher titer and time of induction found, the greater the power of the T-cell modulator.

25. The potentiated T cell modulator according to claim 1, wherein it promotes a significantly increased activity of NK cells (natural killers), which provide protection against viruses as part of the natural immune defense system.

26. Use of potentiated T cell modulator (TCM) with a potency of 1012 leukocytes×mm3 whose specific source is shark spleen said shark spleen rich in B lymphocytes, and is obtained by a dialyzable extract of leukocytes from leukocyte cells containing polypeptides equal to or less than 10,000 daltons, or its pharmaceutically acceptable salt for use as an adjuvant in viral preparations to potentiate vaccines.

27. The use according to claim 13, wherein said adjuvant activity is effective in stimulating the immune response and is devoided of adverse biological properties.
